(19) United States
(12) Patent Application Publication
(10) Pub. No.: US 2003/0055472 A1
(43) Pub. Date: Mar. 20, 2003

(54) Method for supporting medical practitioners in managing patient temperature

(76) Inventor: William J. Worthen, Irvine, CA (US)

Correspondence Address: John L. Rogitz, Suite 3120, 750 "B" Street, San Diego, CA, 92101, US

(21) Appl. No.: 09/956,446

(22) Filed: Sep. 19, 2001

(51) Int. Cl. A61F 7/00

(52) U.S. Cl. 607/105

(57) ABSTRACT

A method for supporting medical practitioners in managing patient temperature includes obtaining regulatory approval to use a heat exchange catheter having a circulating working fluid in a neuro intensive care unit (NICU) to manage patient temperature. Moreover, regulatory approval is obtained to use the catheter outside the NICU for patient temperature management. The catheter can be used outside the NICU to induce therapeutic hypothermia to treat cardiac arrest, stroke, and acute myocardial infarction. Also, the catheter can be used to treat hypothermia after coronary artery bypass graft (CABG) surgery.

### BACKGROUND OF THE INVENTION

1. Field of the Invention

The present invention relates generally to methods and apparatus for exchanging heat with the body of a patient.

2. Description of the Related Art

It has been discovered that the medical outcome for a patient suffering from severe brain trauma or from ischemia caused by stroke or heart attack is improved if the patient is cooled below normal body temperature (37° C.). Furthermore, it is also accepted that for such patients, it is important to prevent hyperthermia (fever) even if it is decided not to induce hypothermia. Moreover, in certain applications such as post-CABG surgery, it might be desirable to rewarm a hypothermic patient.

Having recognized the importance of temperature management, the present invention seeks to support medical practitioners in their endeavors.

### SUMMARY OF THE INVENTION

A method for supporting medical practitioners in managing patient temperature includes obtaining regulatory approval to use a heat exchange catheter having a circulating working fluid in a neuro intensive care unit (NICU) to manage patient temperature. Then, regulatory approval to use the catheter outside the NICU for patient temperature management is obtained.

In a preferred embodiment, regulatory approval is obtained to use the catheter outside the NICU to induce therapeutic hypothermia. Preferably, the therapeutic hypothermia induced via the heat exchange catheter is used to treat cardiac arrest, stroke, and/or acute myocardial infarction. Also, regulatory approval is obtained to use the heat exchange catheter outside the NICU to treat hypothermia after coronary artery bypass graft (CABG) surgery.

In another aspect of the present invention, a method for supporting medical practitioners in managing patient temperature includes obtaining regulatory approval to use a heat exchange catheter having a circulating working fluid in an application not requiring a showing of improved patient outcome. Thereafter, regulatory approval is obtained to use the catheter in an application requiring a showing of improved patient outcome.

In yet another aspect of the present invention, a method for supporting medical practitioners in managing patient temperature includes providing a first central venous catheter that is configured to circulate a working fluid through a heat exchange region. The first catheter is also configured to be inserted into the superior vena cava of a patient through a neck or shoulder entry point. A second central venous catheter that is configured for circulating a working fluid through a heat exchange region is also provided. Moreover, the second catheter is configured to be inserted into the inferior vena cava of a patient through a groin entry point.

In still another aspect of the present invention, a method for treating a patient includes inserting a catheter that has an infusion lumen into a patient's superior vena cava. One or more central venous line functions are undertaken using the catheter. Also, the catheter is connected to a source of heat exchange fluid using a tubing set to circulate the fluid through the catheter to manage patient temperature.

The details of the present invention, both as to its construction and operation, can best be understood in reference to the accompanying drawings, in which like numerals refer to like parts, and which:

### BRIEF DESCRIPTION OF THE DRAWINGS

FIG. 1 is a flow chart of a method for obtaining regulatory approval for use of a heat exchange catheter;

FIG. 2 is a flow chart of an alternative method for obtaining regulatory approval for use of a heat exchange catheter; and

FIG. 3 is a flow chart of a method for using a heat exchange catheter.

### DETAILED DESCRIPTION OF THE PREFERRED EMBODIMENT

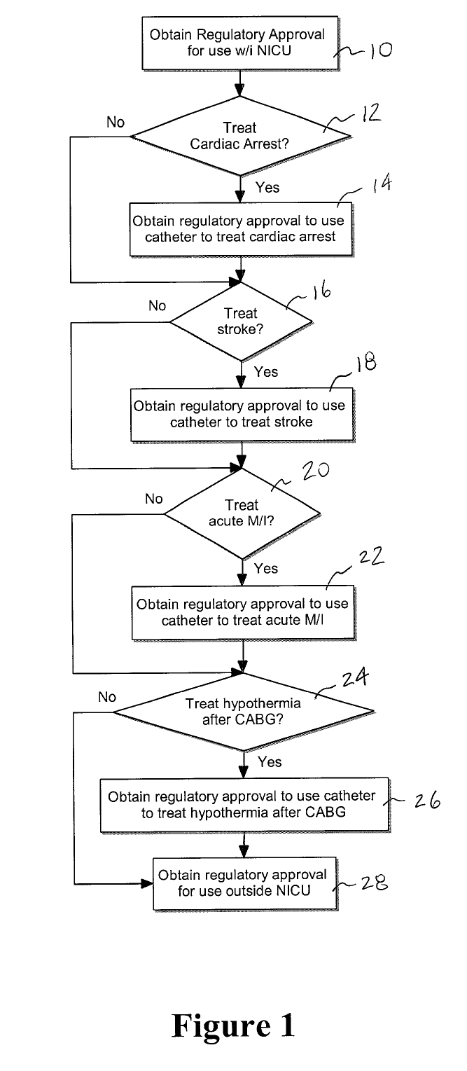

Referring initially to FIG. 1, a method for obtaining regulatory approval for use of a heat exchange catheter is shown and commences at block 10, wherein regulatory approval is obtained for use of the heat exchange catheter within the neuro intensive care unit (NICU) preferably for temperature maintenance, i.e., to prevent unwanted fever, referred to as “hyperthermia,” and maintain patients at normal temperatures (normothermia). Typically, in the U.S., approval to market and use a particular medical device must be obtained with the Food and Drug Administration (FDA) Center for Devices and Radiological Health (CDRH). In a preferred embodiment, the heat exchange catheter is an Alsius catheter, e.g., the indwelling heat exchange catheter disclosed by U.S. Pat. No. 6,126,684 and incorporated herein by reference. Other catheters, including those made Innercool Therapies of San Diego, Calif. and Radiant Medical of Portola Valley, Calif. can also be used.

After filing for regulatory approval to use the heat exchange catheter in the NICU, the method moves to decision diamond 12, wherein it is determined whether the heat exchange catheter is to be used to treat cardiac arrest within the NICU. It is to be understood that this treatment includes inducing hypothermia during the cardiac arrest to alleviate the results of global ischemia. One such method for treating cardiac arrest using hypothermia is disclosed by U.S. Pat. No. 6,149,670, incorporated herein by reference. Returning to decision diamond 12, if it is determined that the catheter is to be used to treat cardiac arrest, the logic moves to block 14 where regulatory approval is obtained to use the catheter in this manner. Thereafter, the logic moves to decision diamond 16, described below.

At decision diamond 12, if the catheter is not to be used to treat cardiac arrest, the logic moves to decision diamond 16 where it is determined whether the catheter is to be used to treat stroke, e.g., by inducing hypothermia immediately after the stroke has occurred to minimize the potential brain damage caused by the stroke. If so, the logic moves to block 18 where regulatory approval is obtained to allow the heat exchange catheter to be used to treat stroke victims. The logic then moves to decision diamond 20, described below. If, at decision diamond 16, it is determined that the catheter is not be used to treat stroke, the logic moves to decision diamond 20 where it is determined whether the heat exchange catheter is to be used to treat acute myocardial infarction (M/I). In one exemplary non-limiting embodiment, the heat exchange catheter can be used to treat acute M/I by inducing hypothermia during the M/I to minimize permanent injury to the patient.

Returning to decision diamond 20, if the heat exchange catheter is to be used to treat acute M/I the logic moves to block 22 where regulatory approval is obtained to use the heat exchange catheter to treat acute M/I. From block 22, the logic moves to decision diamond 24, described below. At decision diamond 20, if it is determined that the heat exchange catheter is not to be used to treat acute M/I, the logic moves to decision diamond 24.

At decision diamond 24, it is determined whether the heat exchange catheter is to be used to treat hypothermia after coronary artery bypass graft (CABG) surgery. During the CABG surgery, the patient is cooled to a hypothermic state in order to slow the patient's heart rate and thus, blood flow, to allow a surgeon to remove a portion of the coronary artery and replace it with a graft taken, e.g., from the patient's leg. After the CABG surgery the patient's temperature must be returned to normal in a controlled manner. This can be achieved through use of the heat exchange catheter.

Returning to decision diamond 24, if it is determined that the heat exchange catheter is to be used to treat hypothermia after CABG surgery, the logic proceeds to block 26 where regulatory approval is obtained that will allow the heat exchange catheter to be used in this manner. The logic then moves to block 28, described below. If, at decision diamond 24, it is determined that the heat exchange catheter is not to be used to treat patients after CABG surgery, the logic moves to block 28 where regulatory approval is obtained to use the heat exchange catheter outside the NICU.

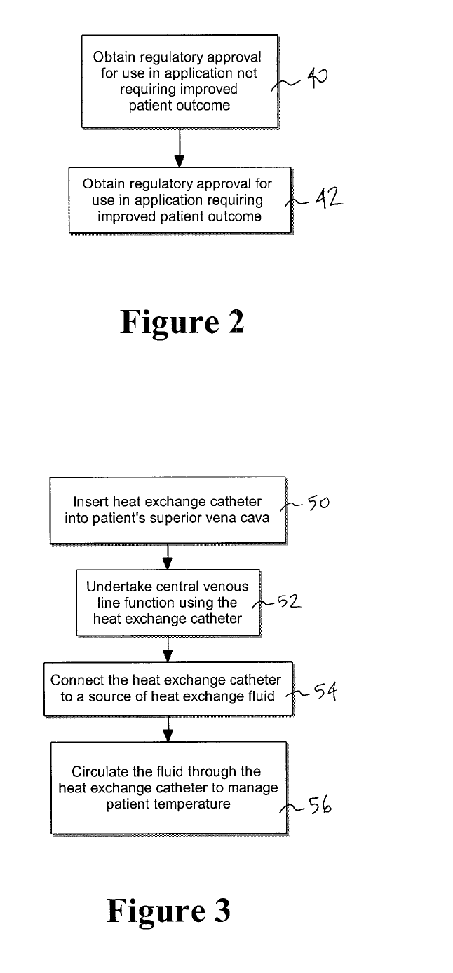

Referring now to FIG. 2, an alternative method for obtaining regulatory approval for using the heat exchange catheter is shown and commences at block 40 where regulatory approval is obtained for use in an application not requiring a showing of improved patient outcome. For example, regulatory approval for a use which is standard of care, e.g., the prevention of fever in the NICU, might not require an endpoint of improved patient outcome, such as an improved Glasgow coma score (GCS), improved Barthel index, or improved NIH stroke scale, but only an endpoint that effective fever control was achieved. Once approval in granted for a clinical endpoint that is something other than improved patient outcome, the logic moves to block 42, where regulatory approval is obtained for use in an application requiring a showing of improved patient outcome, e.g., include inducing hypothermia to treat cardiac arrest, stroke, or myocardial infarction, as described above.

FIG. 3 shows a flow chart of one non-limiting, exemplary method for using a heat exchange catheter. Commencing at block 50, the heat exchange catheter is inserted into the patient's superior vena cava, e.g., through a neck or shoulder entry point. It can be appreciated that the heat exchange catheter can also be inserted into the patient's inferior vena cava, e.g., through a groin entry point. From block 50, the logic moves to block 52 where at least one central venous line function is undertaken using the heat exchange catheter. The central venous line functions can include, e.g., sampling blood, infusing drugs, determining blood pressure, etc. Next, at block 54 the heat exchange catheter is connected to a source of heat exchange fluid. Preferably, the heat exchange catheter is connected to a heat exchange system. U.S. Pat. No. 6,146,411, incorporated herein by reference, discloses one such heat exchange system. Then, at block 56 fluid, e.g., saline, is circulated through the heat exchange catheter to manage patient temperature.

While the particular METHOD FOR SUPPORTING MEDICAL PRACTITIONERS IN MANAGING PATIENT TEMPERATURE as herein shown and described in detail is fully capable of attaining the above-described aspects of the invention, it is to be understood that it is the presently preferred embodiment of the present invention and thus, is representative of the subject matter which is broadly contemplated by the present invention, that the scope of the present invention fully encompasses other embodiments which may become obvious to those skilled in the art, and that the scope of the present invention is accordingly to be limited by nothing other than the appended claims, in which reference to an element in the singular is not intended to mean “one and only one” unless explicitly so stated, but rather “one or more.” All structural and functional equivalents to the elements of the above-described preferred embodiment that are known or later come to be known to those of ordinary skill in the art are expressly incorporated herein by reference and are intended to be encompassed by the present claims. Moreover, it is not necessary for a device or method to address each and every problem sought to be solved by the present invention, for it is to be encompassed by the present claims. Furthermore, no element, component, or method step in the present disclosure is intended to be dedicated to the public regardless of whether the element, component, or method step is explicitly recited in the claims. No claim element herein is to be construed under the provisions of 35 U.S.C. section 112, sixth paragraph, unless the element is expressly recited using the phrase “means for.”

We claim: What is claimed is:

1. A method for supporting medical practitioners in managing patient temperature, comprising:
obtaining regulatory approval to use a heat exchange catheter having a circulating working fluid in a neuro intensive care unit (NICU) to manage patient temperature; and
obtaining regulatory approval to use the catheter outside the NICU for patient temperature management.

2. The method of claim 1, wherein the act of obtaining regulatory approval to use the catheter outside the NICU includes obtaining approval to use the catheter to induce therapeutic hypothermia.

3. The method of claim 2, wherein the act of obtaining regulatory approval to use the catheter outside the NICU includes obtaining approval to use the catheter to induce therapeutic hypothermia to treat cardiac arrest.

4. The method of claim 2, wherein the act of obtaining regulatory approval to use the catheter outside the NICU includes obtaining approval to use the catheter to induce therapeutic hypothermia to treat stroke.

5. The method of claim 2, wherein the act of obtaining regulatory approval to use the catheter outside the NICU includes obtaining approval to use the catheter to induce therapeutic hypothermia to treat acute myocardial infarction

6. The method of claim 1, wherein the act of obtaining regulatory approval to use the catheter outside the NICU includes obtaining approval to use the catheter to treat hypothermia after coronary artery bypass graft (CABG) surgery.

7. A method for supporting medical practitioners in managing patient temperature, comprising:
obtaining regulatory approval to use a heat exchange catheter having a circulating working fluid in an application not requiring a showing of improved patient outcome; and
obtaining regulatory approval to use the catheter in an application requiring a showing of improved patient outcome.

8. The method of claim 7, wherein the act of obtaining regulatory approval to use the catheter in an application requiring a showing of improved patient outcome includes inducing therapeutic hypothermia.

9. The method of claim 8, wherein the act of obtaining regulatory approval to use the catheter in an application requiring a showing of improved patient outcome includes inducing therapeutic hypothermia to treat cardiac arrest.

10. The method of claim 8, wherein the act of obtaining regulatory approval to use the catheter in an application requiring a showing of improved patient outcome includes inducing therapeutic hypothermia to treat stroke.

11. The method of claim 8, wherein the act of obtaining regulatory approval to use the catheter in an application requiring a showing of improved patient outcome includes inducing therapeutic hypothermia to treat acute myocardial infarction.

12. The method of claim 7, wherein the act of obtaining regulatory approval to use the catheter in an application requiring a showing of improved patient outcome includes obtaining approval to use the catheter to treat hypothermia after CABG.

13. A method for supporting medical practitioners in managing patient temperature, comprising:
providing a first central venous catheter configured for circulating a working fluid through at least one heat exchange region, the first catheter being configured for insertion into the superior vena cava of a patient through a neck or shoulder entry point; and
providing a second central venous catheter configured for circulating a working fluid through at least one heat exchange region, the second catheter being configured for insertion into the inferior vena cava of a patient through a groin entry point.

14. The method of claim 13, wherein the first and second catheters each include at least one infusion lumen.

15. The method of claim 13, wherein the first and second catheters each include at least two infusion lumens.

16. The method of claim 13, wherein at least the first catheter is provided with a substrate bearing instructions for inserting the first catheter in the superior vena cava to manage temperature and to undertake central venous line functions.

17. A method for treating a patient, comprising:
inserting a catheter having at least one infusion lumen into a patient's superior vena cava;
undertaking at least one central venous line function using the catheter; and
connecting the catheter to a source of heat exchange fluid using a tubing set to circulate the fluid through the catheter to manage patient temperature.
